(19) United States
(12) Patent Application Publication
(10) Pub. No.: US 2011/0202132 A1
(43) Pub. Date: Aug. 18, 2011

(54) VISUAL PROSTHESIS IMPLANT

(75) Inventor: Peter Bartus Leonard Meijer, Eindhoven (NL)

(73) Assignee: NXP B.V., Eindhoven (NL)

(21) Appl. No.: 13/121,452

(22) PCT Filed: Sep. 16, 2009
(86) PCT No.: PCT/IB2009/054043

§ 371 Date: Mar. 29, 2011

(30) Foreign Application Priority Data
Sep. 29, 2008 (EP) 08165388.3
Sep. 16, 2009 (IB) PCT/IB2009/054043

(51) Int. Cl. A61F 2/16; A61F 2/14

(52) U.S. Cl. 623/6.11; 623/4.1

(57) ABSTRACT

A visual prosthesis implant (1) is provided which comprises an SIMD-based processor array (6) adapted for receiving image signals from an image sensor (5) and outputting processed signals, and a bio-compatible electrode implant (7) receiving the processed signals and adapted for coupling to neurons. Using the SIMD-based processor array (6) provides high performance at small power dissipation and small chip area such that a fully implantable is visual prosthesis is achieved.

### FIELD OF THE INVENTION

The invention relates to a visual prosthesis implant. More particular, the invention relates to a visual prosthesis implant comprising a bio-compatible electrode implant for coupling to neurons.

### BACKGROUND OF THE INVENTION

In recent years, many researchers around the world have been working on retinal implants and cortical implants to restore vision in human patients. In particular, electronic retinal prostheses are being developed for patients affected by common causes of blindness that only damage the light receptors in the retina, such as RP (Retinitis Pigmentosa) and AMD (age-related macular degeneration). Further, cortical implants and deep brain implants for restoring sight to blind people are being developed.

WO 03/090166 A2 discloses a permanent retinal implant device using a NGC array hybridized to a silicon chip, the image being simultaneously generated within each cell through a photon-to-electron conversion using a silicon photodiode.

However, in systems which have been developed thus far, image and electrode processing which is needed to convert live captured video into biocompatible electrode signals is done using either an external PC or equivalent with a general purpose CPU, or using a DSP (digital signal processing unit) or FPGA (field-programmable gate array). However, with respect to the specific envisaged applications, these applied techniques are highly inefficient in terms of power consumption, performance, and chip area. In particular, this inefficiency has frustrated system size reduction and system integration. With respect to a self-contained intraocular implant with an image sensor and an electrode array, the specific circumstances of a very limited available space for electronics and the relatively small acceptable maximum power dissipation have to be considered for the design of a suitable system. For example, the power dissipation should result in heating which is in the order of a tenth of a degree Celsius at maximum to avoid long-term changes in the metabolism.

One solution capable of achieving the required low power dissipation and small area for provision of fully integrated intra-ocular implants which has been considered thus far is based on ASIC (application-specific integrated circuit) design. However, such a design has several drawbacks such as the limited flexibility to program individualized processing as required to account for individual differences between different patients. For example, there are many different causes of blindness, and age also plays an important role such that individual differences necessarily have to taken into account for. Further, electrode coupling to neurons may degrade over time and may vary with respect to individual electrodes from patient to patient. Furthermore, signal processing for electrode stimulation in particular requires high programming flexibility.

### OBJECT AND SUMMARY OF THE INVENTION

It is an object of the present invention to provide a visual prosthesis implant which provides the required low power dissipation, a small size, real-time image processing, and (re-)programming flexibility. Further, the visual prosthesis implant shall allow full integration for e.g. intraocular implantation.

This object is solved by a visual prosthesis implant according to claim 1. The visual prosthesis implant comprises: an SIMD-based processor array adapted for receiving image signals from an image sensor and outputting processed signals, and a bio-compatible electrode implant receiving the processed signals and adapted for coupling to neurons. Thus, the visual prosthesis implant comprises an SIMD-based (single-instruction multiple data) processor array. Processor arrays based on the SIMD processing paradigm provide very high computational efficiency in embedded image processing applications. Thus, low power dissipation, small size, real-time image processing, and programming flexibility can all be simultaneously achieved by making use of on-chip SIMD processing. The high degree of parallelism of n processing cores (with n being an integer) in SIMD processing (a core being typically called a processing element (PE) in SIMD designs) allows for a lower supply voltage for the same performance because only a clock frequency f which is 1/n times the clock frequency of a single core realization is needed, and operation at such a lower clock frequency can be achieved at a lower supply voltage. Although the lower clock frequency f for charging and discharging a chip interconnect to some extent cancels out against the factor n increase in chip area A required for provision of n cores, the power dissipation of a multi-core realization can, for the same performance, still be significantly lower as compared to a single-core system (or a system comprising a few cores). This is due to the fact that the supply voltage contributes quadratically to the power dissipation. As a consequence, the required low power dissipation can be achieved using SIMD-based processor arrays. The parallelism of SIMD processing is also a perfect match to typical low level image processing algorithms in which typically all pixels are subjected to the same set of transformations while any remaining differences in pixel processing can be accounted for through transformation parameters and masking bit vectors. Further, the SIMD architecture offers a large degree of programming flexibility for adapting the performance of the visual prosthesis implant as compared to e.g. an ASIC design. A combination of an image sensor, the SIMD-based processor array, and a bio-compatible electrode array for coupling to neurons offers great advantages over the existing art. In particular, fully integrated intraocular implants can be realized with this combination of features. However, in principle the image sensor can be either provided separate from the visual prosthesis implant or may be integrated in the visual prosthesis implant.

Preferably, the SIMD-based processor array is adapted to output signals conditioned to match bio-compatible electrode characteristics. In this case, only a basic digital-analog converter (DAC) needs to be applied to convert digital signal levels and codes to appropriate voltage, current, and charge injection rates which further helps in reduction of the overall device area and power consumption.

According to an aspect, the SIMD-based processor array is adapted to consume less than 1 mW power. Thus, the power dissipation which might cause heating of surrounding tissue by the visual prosthesis implant is low enough not to cause any long-term changes in metabolism and the visual prosthesis implant is well suited for complete implantation, e.g. as a (completely) intraocular implant.

According to one aspect, the visual prosthesis implant is a high-bandwidth or compute-intensive brain implant or a cortical implant.

According to another aspect, the visual prosthesis implant is an intraocular implant. In this case, the SIMD-based processor array and the electrode implant are adapted to be completely arranged within the eye of the patient and no wiring to the exterior of the eye (which may suffer breakage due the natural eye movements) has to be provided for the stimulation of neurons. Preferably, all electronics of the visual prosthesis implant are adapted for intraocular implantation. In this case, also for image signal processing no wiring to the exterior of the eye is necessary either.

If the visual prosthesis implant comprises an image sensor, all components for restoring visual perception are integrated in one device, namely in the visual prosthesis implant, and thus a particularly small volume suited e.g. for full intraocular implantation can be realized. In this case, the image sensor forms a part of the visual prosthesis implant. Even power-consuming wireless communication is then not needed during normal use, being only needed for more special situations such as when wirelessly changing parameters, or when checking on proper system functioning.

Preferably, the visual prosthesis implant is structured such that the SIMD-based processor array receives digital image signals directly from an image sensor having a digital output or from an analog image sensor via an analog-digital converter. Such an arrangement allows achieving a particularly small overall volume which is advantageous for e.g. intraocular implants.

Preferably, the visual prosthesis implant is adapted to be wirelessly provided with electrical power. In this case, the visual prosthesis implant does not even require wiring to the exterior for power supply, thus reducing risks coming along with external wiring such as possible infections, wiring wear, etc.

According to an aspect, the visual prosthesis implant comprises a low-bandwidth wireless communication unit. A low-bandwidth wireless communication unit also comprises low power dissipation and can e.g. be realized through the zigbee protocol. Such a wireless communication unit may be adapted to allow changing of operation settings of the visual prosthesis implant, uploading new firmware and programs, imposing test images on the electrode array, and reporting device and electrode status or debugging information such that all these features can be realized without requiring wiring to the external.

Further features and advantages will become apparent from the description of an embodiment with respect to the enclosed drawing.

### BRIEF DESCRIPTION OF THE DRAWING

The invention will be described in greater detail hereinafter, by way of non-limiting examples, with reference to the embodiment shown in the drawing.

FIG. 1 is a schematic representation of a visual prosthesis implant which is an intraocular implant in the embodiment.

### DETAILED DESCRIPTION OF THE EMBODIMENT

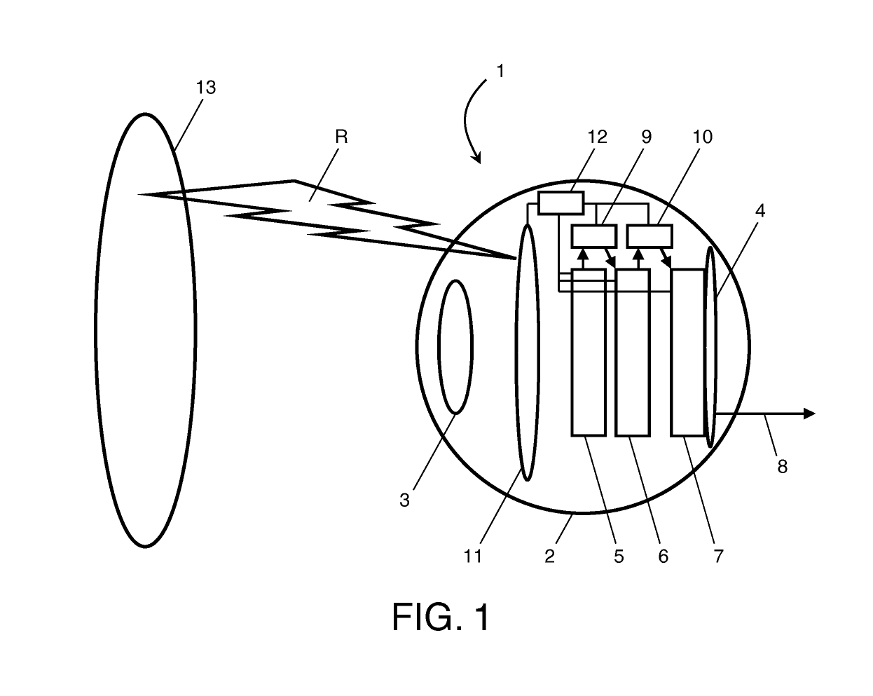

FIG. 1 schematically shows a visual prosthesis implant according to an embodiment which is an intraocular implant. As can be seen in FIG. 1, all components of the visuals prosthesis implant 1 according to the embodiment are provided in the interior of a human eye 2. In the embodiment, all components are situated between the retina 4 and the lens 3. In other embodiments, the electrode array may be positioned behind the retina, as with subretinal implants, and various other components could also be positioned behind the retina. In FIG. 1, a situation is shown in which the lens 3 is the natural lens of the eye 2 and an image sensor 5 is provided in the focal plane of the natural lens 3 near the retina 4. However, instead of using the natural lens 3, image acquisition can also be realized by the image sensor 5 in combination with an artificial lens system. Further, an SIMD-based processor array 6 is provided which is operatively connected to the image sensor 5. The SIMD-based processor array 6 comprises a large number n of processing cores (or processing elements (PE) as they are typically called in SIMD-device design). The processing cores are arranged such that they are capable of performing (substantially identical) operations in parallel. The SIMD-based processor array 6 is further operatively connected to a bio-compatible electrode implant 7 which is adapted to couple to neurons of the retina such that images become visualized by a patient provided with the visual prosthesis implant 1 via an optic nerve 8 (schematically depicted). In the embodiment, the bio-compatible electrode implant 7 may be arranged epi-retinal or sub-retinal. The bio-compatible electrode implant 7 is formed by an electrode array the elements of which are adapted to be coupled to neurons.

An analog-digital converter (ADC) 9 is provided between the image sensor 5 and the SIMD-based processor array 6 for converting analog signals output by the image sensor 5 to digital signals which are suited for the SIMD-based processor array 6. Thus, in this case an analog image sensor 5 is provided. As an alternative, an image sensor 5 having a digital output could be provided such that the ADC 9 can be dispensed with.

A digital-analog converter (DAC) 10 is provided between the SIMD-based processor array 6 and the bio-compatible electrode implant 7 for converting (digital) output signals of the SIMD-based processor array 6 to signals suited for the bio-compatible electrode implant 7. The processed and possibly sub-sampled image signals output by the SIMD-based processor array 6 are conditioned to match bio-compatible electrode characteristics such that only a basic digital-analog converter 10 is needed to convert digital signal levels and codes to appropriate voltage, current and charge injection rates.

The visual prosthesis implant 1 according to the embodiment is thus adapted to sense visual information (optical signals) via the image sensor 5 (outputting either analog or digital signals depending on the type of sensor used), to process the signals by the SIMD-based processor array 6 to generate bio-compatible electrode signals, and to output the generated bio-compatible electrode signals via the bio-compatible electrode implant 7 to achieve visualization.

Depending on the required types of image processing in the SIMD-based processor array 6 (which may be different e.g. for different causes of blindness), a RAM module may be provided (e.g. associated to the SIMD-based processor array 6) to store one or several video frames, or adequate storage may be provided to the SIMD-based processor array itself. The SIMD-based processor array 6 may also be adapted to analyze image content and/or insert informational messages, e.g. through displayed text or warning flashes. Software running on the SIMD-based processor array 6 may also realize adaptive image processing including learning effects to take topological non-idealities into account in the mapping from the bio-compatible electrode implant 7 to visual percepts.

As is further shown in FIG. 1, an electromagnetic receiving coil 11 is provided in the visual prosthesis implant 1 according to the embodiment. The receiving coil 11 is electrically connected to a power converter 12 for converting electromagnetic radiation R (schematically shown in FIG. 1) received by the receiving coil 11 to electrical power which can be used for driving the components of the visual prosthesis implant 1, in particular for driving the image sensor 5, the SIMD-based processor array 6, the bio-compatible electrode implant 7, and the digital-analog converter 10. In order to provide the electrical power to the various components, the power converter 12 is connected to the image sensor 5, the SIMD-based processor array 6, the bio-compatible electrode implant 7, and the ADC 9 and the DAC 10 via schematically indicated wiring. The electromagnetic radiation can for instance be provided by an external coil 13 which is also schematically shown in FIG. 1. Electrical power supply may be buffered through a rechargeable battery or capacitor such that proper device operation can continue even while the electromagnetic power input is interrupted, possibly extending to a full day of use or longer before recharging.

As an alternative to the magnetic coupling via coils described above, power to the visual prosthesis implant 1 can also be provided via an external light source (or a bright head-mounted display) shining into the eye and intraocular energy scavenging based on the photovoltaic effect. As a skilled person understands, in this case different internal components (instead of the receiving coil 11 and the power converter 12) have to be provided for provision of the required electrical energy within the visual prosthesis implant.

Since all electronics of the visual prosthesis implant according to the embodiment are intraocular without any percutaneous wiring, the risk of infections and the risk of breaking wires due to natural eye movements are adequately minimized.

According to a particularly preferred embodiment, a low-bandwidth (and hence low-power consumption) wireless communication unit may be additionally provided in the visual prosthesis implant 1. In this case, the wireless communication unit is connected to the SIMD-based processing array 6 and other components of the visual prosthesis implant. Wireless communication can e.g. be realized through the zigbee protocol. Using the wireless communication unit, for instance operating settings of the SIMD-based processing array 6 can be changed, and new (or different) firmware and programs can be uploaded. As a further possibility, test images can be imposed on the bio-compatible electrode implant 7 and/or the device and electrode status can for instance be reported to an external device and/or debugging information in case of operation problems can be provided.

It has been described above that the SIMD-based processor array 6 comprising n processing cores is provided for processing the information provided by the image sensor 5 to generate bio-compatible electrode signals. In the following, the advantages of using such a SIMD-based processor array 6 as compared to known systems will be described.

As compared to known systems, the high degree of parallelism of n processing cores in the SIMD processing allows using a lower supply voltage for achieving the same performance as compared to visual prosthesis implants based on a single core (or only a few cores operating in parallel). This is based on the fact that a clock frequency f can be used which is only 1/n times the clock frequency required for processing in a single core. Processing at the lower clock frequency f can be achieved using a lower supply voltage. Although the lower clock frequency f for charging and discharging the (CMOS) interconnect present in the visual prosthesis implant 1 to some extent cancels out against an increase in overall chip area A required for realizing n processing cores, the power consumption (and thus power dissipation) will nevertheless be significantly lower for achieving the same performance, since the supply voltage contributes quadratically to the power dissipation.

An example of a SIMD-based processor array is the 320-core Xetal-I chip having a chip size of 22 mm² which offers a performance of up to 5 GOPS (Giga-operations per second) or dissipates 30 mW for typical conventional image processing applications. Another example of a SIMD-based processor array is the 128-core IMAP. Both examples for SIMD-based processor arrays are essentially 1-D arrays (wherein 1-D is not to be understood absolutely quantitatively but rather qualitatively with e.g. an arrangement in a 128×2 array or in a 128×4 array still being called 1-dimensional as compared to rather square organizations such as a 128×128 array or a 320×240 array which would be called 2-dimensional). With respect to such 1-D arrays, it has been found that these offer no less computational and data I/O efficiency as compared to (rather square) 2-D arrays. With respect to applications for visual prosthesis implants, for visual prostheses with modest resolution, the processing speed of these SIMD-based processor arrays can be throttled back by using a much lower clock frequency which results in a significant reduction in power consumption (and dissipation). For example, the Xetal-I SIMD-based processor array can be operated down to the microwatt range (μW) before current leakage limits further power reduction. Thus, such a SIMD-based processor array can be operated such that image and electrode processing can be performed within the eye (in an intraocular implant) where tissue heating provides a power dissipation constraint requiring power to stay below 1 mW to avoid a biologically significant temperature rise.

Furthermore, the parallelism in SIMD processing is also a perfect match to typical low level image processing applications in which typically all pixels are subjected to the same set of transformations while (any remaining) differences in pixel processing can be accounted for through transformation parameters and masking bit vectors. Typical transformations and image enhancement operations suiting a SIMD-based processor array include (without being limited to this): Gaussian filtering (blurring), edge detection, edge enhancement, corner detection, motion detection and tracking, color segmentation, histogram generation, thresholding and other non-linear mappings.

According to the described embodiment, the visual prosthesis implant is adapted such that each (down-sampled) pixel in principle maps to a single electrode, although neighborhood processing will aid with contrast enhancement and various other perceptual (un-)masking issues in generating phosphenes. The actual transformation to be applied to all pixels and electrodes (of the bio-compatible electrode implant 7) will likely change over the time the visual prosthesis implant is used, either due to improved insights into the required transformations for optimum perceptual results in the future or e.g. because of individual differences between different patients. Further, it is likely that the actual transformations have to be changed due to adaptation of the patient to the prosthesis and/or because the respective situation in which the visual prosthesis implant is used requires a dedicated mapping (e.g. different mapping for rather stationary use such as reading and mobile use such as walking). It is a further advantage of the proposed SIMD processing that changing the transformations to be applied can efficiently be realized through uploading programs and parameters for all SIMD cores in common (e.g. in a global controller RAM or flash memory which can be provided in the visual prosthesis implant). In contrast, an ASIC would not nearly offer the same level of programming flexibility.

Further, today optimum stimulus signals (including wave shapes) for implanted electrodes are not yet known and it is very likely that the stimulus signals will have to be adapted in the future. Again, the SIMD-based processor array offers the required ability to be programmed and thus the required flexibility.

Further, if any higher level algorithms are required which are not of parallel nature, they can e.g. be mapped to a global controller (which for instance may also dispatch instructions to the SIMD-based processor array). This global controller can take the form of one or more low-power general purpose CPU cores, e.g., ARM-based, and apart from applying higher level algorithms to a much reduced data set it also supports the various control and communication tasks while the SIMD-based processor array takes care of most of the parallelizable compute-intensive processing and streaming data tasks. This global controller may run either continuously or be invoked only when its services are needed, e.g., to significantly save power. As an alternative, the supplementary tasks may also be handled by a simple low-power microcontroller which does not need to provide high performance (e.g. an 8051 microcontroller).

Thus, according to the embodiment described above, image and electrode signal processing for a visual prosthesis is rendered implantable by application of SIMD processing at low power (in particular below 1 mW), with high performance (e.g. 5 GOPS/W or 50 MOPS at 1 mW, or at 1 mW: 400 operations per pixel at a 64×64 resolution and 30 fps which can be achieved with the above mentioned Xetal-I SIMD-based processing array), and with small chip area.

The application of the visual prosthesis implant which has been described with respect to the embodiment is an intraocular implant. However, the invention is not limited to this and cortical implants and other types of high-bandwidth or compute-intensive brain implants (e.g. relating to deep brain implant visual prostheses that interface to the lateral geniculate nucleus (LGN) in the thalamus) are further areas of application.

For the proposed visual prosthesis applications, the processor array lengths can be adapted different to the known SIMD-based processor arrays mentioned above such that the trade-off between performance and power dissipation can be even better adjusted to the specific requirements in visual prosthesis implants. For example, a SIMD-based processor array comprising 64 cores as a perfect match to a 64×64 pixel implant is possible which achieves a significant reduction in chip area as compared to the above mentioned SIMD-based processor arrays. The achievable size reduction may also result in specific SIMD-based processor arrays for visual prosthesis implants which are small enough to be put over the electrode matrix in an intraocular implant without occluding remaining or intact peripheral vision (a situation which is typical for AMD). As a further alternative, the processor may be placed outside the optical paths of the eye, for instance near the front of the eye close to the iris.

Although it is preferred in view of the risk of infections and the risk of breakage of wiring due to natural eye movements that the image sensor forms a part of the visual prosthesis implant, it should be noted that the invention is not limited to this and an external image sensor could also be used.

1. Visual prosthesis implant comprising:
a SIMD-based processor array adapted for receiving image signals from an image sensor and outputting processed signals, and
a bio-compatible electrode implant receiving the processed signals and adapted for coupling to neurons.

2. Visual prosthesis implant according to claim 1, wherein the SIMD-based processor array is adapted to output signals conditioned to match bio-compatible electrode characteristics.

3. Visual prosthesis implant according to claim 1, wherein the SIMD-based processor array is adapted to consume less than 1 mW power.

4. Visual prosthesis implant according to claim 1, wherein the visual prosthesis implant is one of a high-bandwidth or compute-intensive brain implant and a cortical implant.

5. Visual prosthesis implant according to claim 1, wherein the visual prosthesis implant is an intraocular implant.

6. Visual prosthesis implant according to claim 5, wherein all electronics of the visual prosthesis implant are adapted for intraocular implantation.

7. Visual prosthesis implant according to claim 1, further comprising an image sensor.

8. Visual prosthesis implant according to claim 1, wherein the visual prosthesis implant is structured such that the SIMD-based processor array receives digital image signals one of directly from an image sensor having a digital output and from an analog image sensor via an analog-digital converter.

9. Visual prosthesis implant according to claim 1, wherein the visual prosthesis implant is adapted to be wirelessly provided with electrical power.

10. Visual prosthesis implant according to claim 1, wherein the visual prosthesis implant further comprises a low-bandwidth wireless communication unit.
